(19) United States
(12) Patent Application Publication
(10) Pub. No.: US 2009/0118760 A1
(43) Pub. Date: May 7, 2009

(54) Vascular filter retrieval device

(75) Inventors: Jacob Lund Clausen, Kgs. Lyngby (DK); Per Hendriksen, Herlufmagle (DK)

Correspondence Address: COOK GROUP PATENT OFFICE, P.O. BOX 2269, BLOOMINGTON, IN, 47402, US

(73) Assignees: William Cook Europe ApS, Bjaeverskov (DK); Cook Incorporated, Bloomington, IN (US)

(21) Appl. No.: 11/983,152

(22) Filed: Nov. 7, 2007

(51) Int. Cl. A61M 29/00

(52) U.S. Cl. 606/200

(57) ABSTRACT

A vascular filter retrieval device comprising a catheter and a loop for engaging a filter is described. The loop is extendable from a proximal end of the catheter and comprises a resilient wire. The wire may be a metal alloy such as an ELGILOY® alloy. The loop is typically radiopaque and may have a platinum or palladium wire coiled around at least a part thereof. Also described is a method of retrieving a vascular filter from a patient using the device. The catheter is introduced into the vena cava of a patient adjacent the filter. The loop is advanced so that it extends from the proximal end of the catheter and surrounds the filter. The loop is then engaged with a hook on the filter and the device and filter removed from the patient.

### TECHNICAL FIELD

The present invention relates to a vascular filter retrieval device and to a method for retrieving vascular filters implanted in patients, and particularly to the retrieval of filters from the vena cava.

### BACKGROUND OF THE INVENTION

Vascular filters are used for effective filtration of blood particularly in the inferior vena cava in order to prevent pulmonary thromboembolism. Filters typically have a conical design provided with a hook on a top surface for accurate placement in the vena cava. A filter is introduced into a patient using a standard percutaneous technique. If a patient's risk of thromboembolism diminishes, the patient no longer requires a filter and the filter can be removed. A known retrieval device comprises a snare, shaped as a loop, which is used to catch the hook in the top of the filter. The loop is made of seven twisted platinum wires. A problem with this device is that the loop can lose its shape during the attempt to engage the hook of the filter. This necessitates the removal of the loop which then has to be reshaped and cleaned before the retrieval procedure is repeated. There is a strong risk of the loop collapsing if the resistance in the vessel is high. Accordingly, it is not suitable for the secure retrieval of a vascular filter.

### SUMMARY OF THE INVENTION

The present invention seeks to provide an improved vascular filter retrieval device and a method for retrieving vascular filters.

According to a first aspect of the present invention there is provided a vascular filter retrieval device comprising a catheter, said catheter having a proximal end, an expandable loop extendable from said proximal end for engaging a filter, wherein said loop comprises a resilient wire biased towards an expanded position.

An advantage of this device is that as the loop comprises a resilient wire biased towards an expanded position, the radial force produced by the wire when compressed in the catheter is higher than the prior art device and so the risk of the loop collapsing is reduced. The resilient nature of the wire ensures the loop maintains a predetermined shape and size, and so it is easier to locate and snare a vascular filter. Moreover, the device provides a secure grip for retrieval of a filter.

In an expanded position, loop has a width which is typically similar to or the same as the width of the vessel in which the device is intended to be used.

In an embodiment, the resilient wire is heat treated to increase the radial force. Typically, the resilient wire is heated to about 200° C. Alternatively, the resilient wire is cold worked to increase the radial force produced by the wire when this is compressed.

The resilient wire may comprise a metal alloy, for example a Cobalt Chromium Nickel alloy such as ELGILOY®.

The loop may comprise a radiopaque material. A further wire may be coiled around at least a part of the loop. Preferably, the wire is coiled around the entire loop for ease of manufacture. The coiled wire may be platinum for high radiopacity to improve visualisation during retrieval. Alternatively, other radiopaque materials may be used such as palladium. The catheter may be radiopaque.

According to a second aspect of the present invention there is provided a method of removing a vascular filter from a vena cava of a patient comprising the steps of providing a vascular filter retrieval device comprising a catheter, said catheter having a proximal end, a loop extendable from the proximal end and wherein said loop comprises a resilient wire, introducing the catheter into the vena cava adjacent the filter, advancing the loop so that it extends from the proximal end of the catheter and surrounds the filter, engaging the loop with a hook on the filter and removing the device and filter from the patient.

### BRIEF DESCRIPTION OF THE DRAWING

Preferred embodiments of the invention will now be described by way of example only and with reference to the drawings in which:

FIG. 1 is a schematic view of the loop of the vascular retrieval device;

FIG. 2 is a schematic view of a retrieval system comprising the device; and

FIG. 3a to 3e is a schematic view of the device in use.

### DETAILED DESCRIPTION

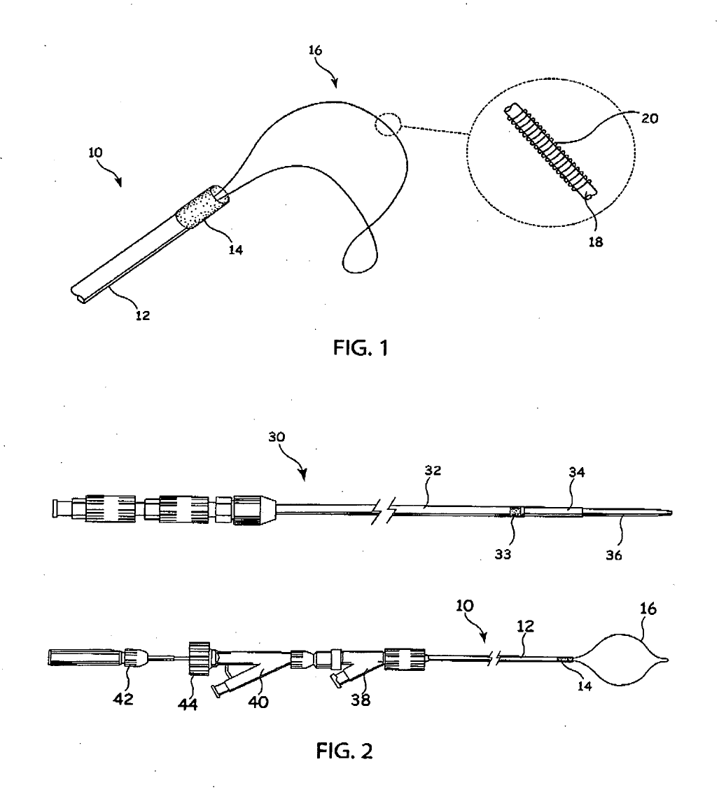

Referring to FIG. 1, the preferred embodiment of device 10 for retrieving a vena cava filter 50 comprises a catheter 12. A wire loop 16 extends through the catheter 12 towards and out of the proximal end 14 of the catheter 12. The wire loop 16 comprises a spring wire 18 made of a metal alloy such as an ELGILOY® alloy. ELGILOY® (also known as phynox) is a Cobalt-Chromium-Nickel alloy. The typical chemical composition of the alloy is 39-41% Cobalt, 19-21% Chromium, 14-16% Nickel and 6-8% Molybdenum. ELGILOY® gives a combination of high strength, ductility and good mechanical properties. It has excellent fatigue life, corrosion resistance in numerous environments and is non magnetic.

The spring wire is substantially covered by a platinum coil 20 wrapped around the spring wire 18. During manufacture of the device, the spring wire 18 is heat treated (200° C. for 8 hours). The proximal end 14 of the catheter 12 has a radiopaque band.

When the loop 16 is fully extended from the proximal end 14 of the catheter 12, it expands to a predetermined shape and size. The size is predetermined so that the loop 16 expands to, or proximate, the full width of the vena cava. The predetermined shape of the loop 16 is circular or lemon shaped.

As shown in FIG. 2, the distal end of the catheter 12 is connected to a side arm adapter 38 and a lockable clear Y fitting 40. The distal end of the wire 16 extends beyond the Y fitting 40 to a pin vise 42.

The device can be used with a known retrieval system comprising a coaxial retrieval sheath system 30 which includes a radiopaque sheath 32 with a radiopaque band 33, and inner 36 and outer 34 radiopaque coaxial catheters, an entry needle, a wire guide and a dilator.

In a particular medical treatment, the Y fitting 40 is held and the pin vise 42 is pulled back so that the loop 16 is covered by the catheter 12 during insertion of the device into the patient. The screw 44 of the Y fitting 40 is tightened to keep the loop 16 within the catheter 12. The right jugular vein of a patient is punctured using the Seldinger technique. Although the right jugular vein approach is preferred, approach via the left jugular vein is possible.

A flush catheter is positioned inferior to the filter 50 and a diagnostic vena cava performed. The flush catheter is then exchanged for the coaxial retrieval sheath system 30 by advancing this over the wire guide. The inner coaxial catheter 36 and the wire guide are removed once the retrieval shaft system 30 is in place. The position of the system can be verified by injection of contrast medium.

The device 10 is then introduced through the coaxial retrieval sheath system 30. The side arm adapter 38 of the device 10 is advanced and connected to the sheath system 30. The adapter 38 can be tightened around the catheter to prevent loss of blood. The screw of the Y fitting 40 is then loosened to enable advancement of the loop 16 inside the catheter 12.

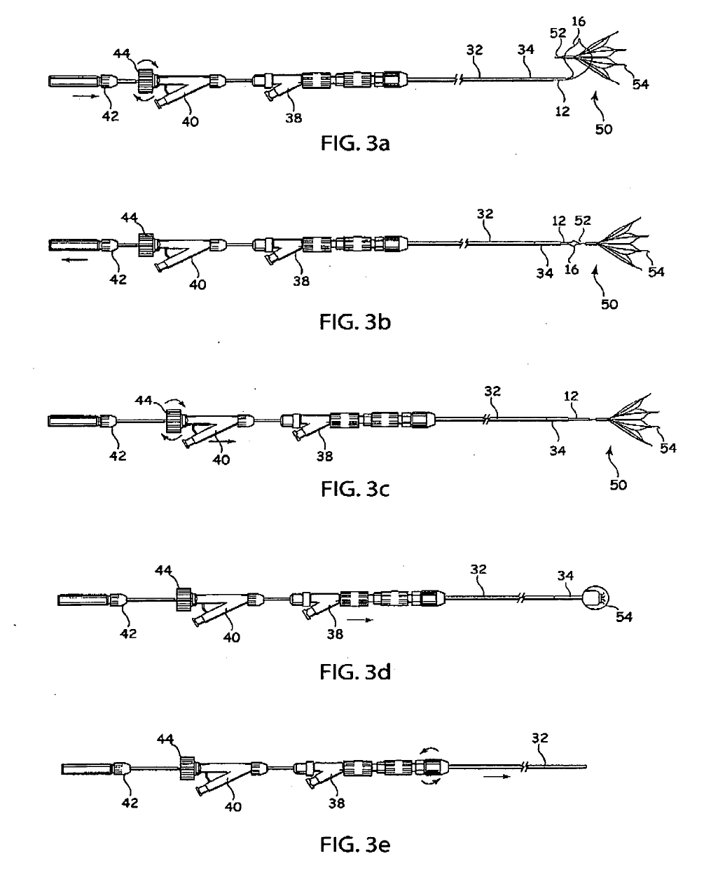

Whilst holding the Y fitting 40, the pin vise 42 is pushed forward and advanced until the loop 16 has fully expanded inside the vena cava and surrounds the filter 50 (FIG. 3a).

As the loop 16 is resilient, it expands to a predetermined shape and size. This is preferably a predetermined size so that it expands to the full width of the vena cava. Accordingly, when the loop 16 is advanced, any object in the vena cava will be snared. This allows for easier and more accurate retrieval. Furthermore, the resilience of the loop ensures that the loop 16 does not lose its shape as it is advanced towards the hook 52 and during engagement thereof. Accordingly, it is not necessary to remove the loop for reshaping and cleaning and the retrieval procedure does not need to be repeated. Thus the retrieval procedure is much quicker.

The loop 16 is then pulled back until it engages the hook 52 of the filter 50 (FIG. 3b). The wire loop 16 is held steady with the pin vise 42, and the Y fitting 40 is pushed with the catheter 12 forward until it touches the hook 52 of the filter 50 (FIG. 3c).

In order to snare the filter 50 in this position, the screw 44 of the Y fitting 40 is firmly locked on the wire loop 16.

Holding the device 10 steady with Y fitting 40, the side arm adapter 38 and outer catheter 34 of the coaxial retrieval sheath system 30 is advanced. The outer catheter 34 is advanced over the filter 50 to collapse it, disengaging the barbed hooks 54 of the filter 50 from the caval wall (FIG. 3d). When the tip of the coaxial retrieval sheath system 30 is at the barbed hooks 54, the hub of the outer sheath 32 is loosened, and the outer sheath 32 is advanced to cover the whole filter 50 including the barbed hooks 54 (FIG. 3e). The complete assembly can then be retrieved.

Although specific embodiments have been described above the invention is not to be limited thereto but is to be defined by the appended claims.

What is claimed is:

1. A vascular filter retrieval device comprising a catheter having a proximal end, an expandable loop for engaging a filter, said loop extendable from the proximal end, wherein said loop comprises a resilient wire biased towards an expanded position.

2. A vascular filter retrieval device as claimed in claim 1 wherein the resilient wire is one of: heat treated and cold worked to increase the radial force produced by the wire when this is compressed.

3. A vascular filter retrieval device as claimed in claim 1 wherein the resilient wire comprises a metal alloy.

4. A vascular filter retrieval device as claimed in claim 3 wherein the metal alloy is a cobalt chromium nickel alloy.

5. A vascular filter retrieval device as claimed in claim 1 wherein a further wire is coiled around at least a part of the loop.

6. A vascular filter retrieval device as claimed in claim 5 wherein the wire is one of platinum and palladium.

7. A vascular filter retrieval device as claimed in claim 1 wherein the loop is radiopaque.

8. A vascular filter retrieval device as claimed in claim 1 wherein the catheter is radiopaque.

9. A vascular filter retrieval device comprising a catheter having a proximal end, a wire loop for engaging a filter, said loop extending through the catheter and extendable from the proximal end, wherein said loop comprises a metal alloy spring wire covered by a coil thereon, the coil being one of platinum and palladium.

10. A method of removing a vascular filter from a vena cava of a patient comprising the steps of:
providing a vascular filter retrieval device comprising a catheter, said catheter having a proximal end, a loop extendable from the proximal end wherein said loop comprises a resilient wire,
introducing the catheter into the vena cava adjacent the filter,
advancing the loop so that it extends from the proximal end of the catheter and surrounds the filter,
engaging the loop with a hook on the filter and removing the device and filter from the patient.
